(19) United States
(12) Patent Application Publication
(10) Pub. No.: US 2026/0260723 A1
(43) Pub. Date: Sep. 3, 2026

(54) SYSTEMS AND METHODS FOR SENTIMENT ANALYSIS OF PATIENT DATA

(71) Applicant: metricHEALTH Solutions Inc., Barrie (CA)

(72) Inventors: Ross Bigelow, Barrie (CA); Chris Adams, Barrie (CA)

(21) Appl. No.: 19/553,500

(22) Filed: Mar. 2, 2026

Related U.S. Application Data
(60) Provisional application No. 63/765,204, filed on Feb. 28, 2025.

(51) Int. Cl. G16H 10/60

(52) U.S. Cl. CPC G16H 10/60

(57) ABSTRACT

Provided are systems and methods for analyzing at least one sentiment of user data, particularly patient data. A computer system for analyzing a sentiment of user data includes at least one processor configured to execute processor-executable instructions stored on at least one non-transitory memory to implement a sentiment analysis engine configured to receive user data from at least one source and at least user, analyze the user data to identify a sentiment of the user data, for at least one sentiment target, based on keywords and language found within the user data, using at least one artificial intelligence (AI) model, and generate at least one output indicating the sentiment of the user data for the at least one sentiment target. The sentiment analysis engine may be used for multiple applications including analyzing sentiment for pharmaceuticals and providing outputs regarding drug adverse reactions, drug efficacy, and likelihood of patient adherence.

### TECHNICAL FIELD

The embodiments disclosed herein relate to processing of patient-derived data to generate insights and, in particular to analyzing sentiments of input qualitative and quantitative patient data to derive insights and predict outcomes.

### Introduction

Currently, patient data which is collected during healthcare delivery, e.g., for pharmaceutical trials, is analyzed quantitatively to determine adverse reactions, treatment satisfactions, patient adherence, etc. Existing tools for analyzing such patient data rely on structured data or limited natural language processing applications, often analyzing data from a single data stream (e.g. behavioral or physiological), to generate limited insights. The lack of qualitative data or data which is integrated from multiple sources and/or data streams results in limitations on the amount and quality of insights which can be gleaned from patient data, resulting in poorer healthcare outcomes for both individual patients as well as other stakeholders (e.g. healthcare providers, pharmaceutical companies, insurance companies, etc.) than could be achieved if available data was analyzed at a qualitative level.

Accordingly, there is a need for systems and methods which employ tools which can analyze qualitative data from multiple data streams to generate better insights and to achieve better healthcare outcomes.

### SUMMARY

Provided herein is a computer system for analyzing a sentiment of user data, the system including at least one processor configured to execute processor-executable instructions stored on at least one non-transitory memory to implement a sentiment analysis engine configured to receive user data from at least one source and at least user, analyze the user data to identify a sentiment of the user data, for at least one sentiment target, based on keywords and language found within the user data, using at least one artificial intelligence (AI) model, and generate at least one output indicating the sentiment of the user data for the at least one sentiment target.

The user data may be received from multiple unstructured data sources.

The user data may be patient data.

The user data may be for a single user.

The user data may be for a cohort of users.

The at least one source may include at least one of electronic health records (EHRs), surveys, clinical notes, social media, interactions with AI assistants, interactions with educational applications, insurance claims, healthcare platforms, emails, and text messages.

The at least one AI model may include at least one of a machine learning model and a natural language processing model.

The at least one sentiment target may be at least one of: a pharmaceutical, a medical therapy, educational materials, an AI assistant, policies and regulations, an insurance claim, a healthcare platform, mental health, and a health program campaign.

The at least one output may include a sentiment analysis index score for at least one sentiment target.

The sentiment analysis index score may be numerical.

The sentiment analysis index score may be one of positive, negative, or neutral.

Multiple sentiment analysis index scores may be provided for each sentiment target. Each sentiment analysis score may represent a specific demographic subset of the user data. The demographics may include at least one of: sex, gender, age, ethnicity, and medical history.

The at least one output may include predictive analytics for an outcome associated with the sentiment target.

The predictive analytics may provide predictions for at least one of: patient adherence, drug efficacy, adverse drug reactions, compliance, and fraud detection.

The at least one output may include a report providing suggestions for next steps regarding the sentiment target.

The report may provide next steps for at least one of: improving educational materials, improving communication, improving treatment recommendations, improving compliance, improving healthcare platforms, interventions for mental health, and improving health program campaigns.

Provided herein is a method of analyzing a sentiment of user data by a sentiment analysis engine comprising at least one processor and at least one non-transitory memory storing processor-executable instructions, the method including receiving, at the sentiment analysis engine, user data from at least one source and at least user, analyzing, by at least on AI model, the user data to identify a sentiment of the user data for at least one sentiment target, based on keywords and language found within the user data, and generating at least one output indicating the sentiment of the user data for the at least one sentiment target.

The user data may be received from multiple unstructured data sources.

The user data may be patient data.

The user data may be for a single user.

The user data may be for a cohort of users.

The at least one source may include at least one of electronic health records (EHRs), surveys, clinical notes, social media, interactions with AI assistants, interactions with educational applications, insurance claims, healthcare platforms, emails, and text messages.

The at least one AI model may include at least one of a machine learning model and a natural language processing model.

The at least one sentiment target may be at least one of: a pharmaceutical, a medical therapy, educational materials, an AI assistant, policies and regulations, an insurance claim, a healthcare platform, mental health, and a health program campaign.

Generating the at least one output may include generating a sentiment analysis index score for at least one sentiment target.

The sentiment analysis index score may be numerical.

The sentiment analysis index score may be one of positive, negative, or neutral.

Multiple sentiment analysis index scores may be provided for each sentiment target. Each sentiment analysis score may represent a specific demographic subset of the user data. The demographics may include at least one of: sex, gender, age, ethnicity, and medical history.

Generating the at least one output may include generating predictive analytics for an outcome associated with the sentiment target.

The predictive analytics may provide predictions for at least one of: patient adherence, drug efficacy, adverse drug reactions, compliance, and fraud detection.

Generating the at least one output may include generating a report providing suggestions for next steps regarding the sentiment target.

The report may provide next steps for at least one of: improving educational materials, improving communication, improving treatment recommendations, improving compliance, improving healthcare platforms, interventions for mental health, and improving health program campaigns.

Other aspects and features will become apparent to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is an example computer network system for sentiment analysis of patient data, according to an embodiment;

FIG. 2 is a block diagram of a computing device, according to an embodiment;

FIG. 3 is a block diagram showing the flow of data through a sentiment analysis system, according to an embodiment;

FIG. 4 is a block diagram of possible application of a sentiment analysis system, according to an embodiment;

FIG. 5 is a flow diagram of a method employing a sentiment analysis engine to provide insights from patient data, according to an embodiment;

FIG. 6 is a flow diagram of a method for a sentiment-based pharmacovigilance system using a sentiment analysis engine to analyze a sentiment of pharmacovigilance patient data, according to an embodiment;

FIG. 7 is a flow diagram of a method for predictive patient adherence using a sentiment analysis engine to analyze a sentiment of patient data together with wearable device data, according to an embodiment;

FIG. 8 is a flow diagram of a method 800 for a dynamic sentiment-based health literacy tool using a sentiment analysis engine to analyze a sentiment of patient/user data regarding health educational material, according to an embodiment;

FIG. 9 is a flow diagram of a method 900 for a virtual sentiment-aware telehealth assistant using a sentiment analysis engine to analyze a sentiment of patient/user data generated during interaction with an AI telehealth assistant, according to an embodiment;

FIG. 10 is a flow diagram of a method 1000 for real-time regulatory feedback monitoring using a sentiment analysis engine to analyze a sentiment of patient (or members of the public) data regarding healthcare policies and regulations (e.g., vaccine schedules), according to an embodiment;

FIG. 11 is a flow diagram of a method 1100 for sentiment-guided fraud detection for insurance claims using a sentiment analysis engine to analyze a sentiment of a patient filing an insurance claim, according to an embodiment;

FIG. 12 is a flow diagram of a method 1200 multi-modal real-world evidence analysis using a sentiment analysis engine to analyze a sentiment of patient data from electronic health records and at least one wearable device, according to an embodiment;

FIG. 13 is a flow diagram of a method 1300 of using a sentiment analysis engine to analyze a sentiment of patient data related to a gamified health platform for a patient prescribed a specific therapy, according to an embodiment;

FIG. 14 is a flow diagram of a method 1400 for AI-driven mental health risk detection using a sentiment analysis engine to analyze a sentiment of patient data, according to an embodiment;

FIG. 15 is a flow diagram of a method 1500 for sentiment-based competitive health campaign analysis using a sentiment analysis engine to analyze a sentiment of patient data for multiple health campaigns, according to an embodiment.

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

Provided herein are systems and methods for sentiment analysis of patient data using AI-based tools, such as machine learning or natural language processing algorithms.

A sentiment analysis engine takes as input multi-patient data from a plurality of sources and uses machine learning or natural language processing algorithms to determine an overall sentiment for at least one sentiment target. Herein “sentiment” represents the general attitude and/or opinion (either conscious or subconscious) of a patient or user, or groups of patients or users towards a specific sentiment target. The sentiment target may be a pharmaceutical or a specific treatment plan. While herein, the sentiment target is most often described as a drug or pharmaceutical, it is to be understood that the sentiment target could be anything which a patient is interacting with. It is also to be understood that the sentiment analysis may be applied to non-patients, for example customers or consumers of services.

The patient data input may include data from patient surveys, social media, electronic health records (EHRs), etc. The sentiment analysis engine identifies patterns in the data of patient sentiment, categorizes patient feedback into key themes, and delivers outputs including predictive analytics such as dissatisfaction or treatment non-adherence.

The sentiment analysis engine can be integrated with healthcare systems or other systems, to both receive data and provide outputs through graphic user interfaces, e.g., visual dashboards, and APIs (application programming interfaces). The systems and methods described herein can empower healthcare providers to make informed, data-driven decisions, ultimately improving patient engagement, clinical outcomes, and operational efficiency.

A specific application of the sentiment analysis systems and methods described herein is to analyze patient data to find adverse drug reactions (ADRs). In such an embodiment the system identifies and categorizes sentiments to detect early signals of potential ADRs and/or medication inefficacy. Through real-time analysis and predictive modeling, sentiment analysis offers actionable insights to pharmaceutical companies and regulatory authorities, enabling proactive risk mitigation and compliance with pharmacovigilance standards. This capability also allows researchers and healthcare providers to evaluate drug performance, detect safety signals, and assess unmet medical needs beyond controlled clinical trial environments.

The key features of the systems and methods described herein include:
i) Sentiment Analysis Engine, which detects emotional tone, satisfaction levels, and concerns in patient communications and data, and distinguishes between positive, negative, and neutral sentiments. The data may be analyzed in real-time, with new data being analyzed as it is provided to the system, enabling timely generation of insights to prevent (further) adverse outcomes for patients.
ii) Patient Feedback Categorization, wherein patient feedback/data is classified into actionable categories (e.g., treatment satisfaction, service experience, or side-effect concerns), and patterns and recurring issues are identified for targeted improvements.
iii) Dynamic Reporting and Insights, which generates visual and interactive dashboards for healthcare administrators and clinicians, and offers trend analyzes to track patient satisfaction over time and across departments.
iv) Predictive Analytics, which uses historical sentiment data to forecast potential risks, such as patient dissatisfaction or treatment non-compliance, and provides recommendations for preemptive measures to improve patient care.
v) Integration Capabilities, which seamlessly integrate with EHRs, patient portals, and communication platforms to consolidate data from multiple sources, and provides APIs for connection with custom healthcare applications.
vi) Privacy and Compliance, which ensures data security and compliance with healthcare regulations like Health Insurance Portability and Accountability Act (HIPAA) and General Data Protection Regulation (GDPR), and employs anonymization techniques to protect patient identities during analysis.

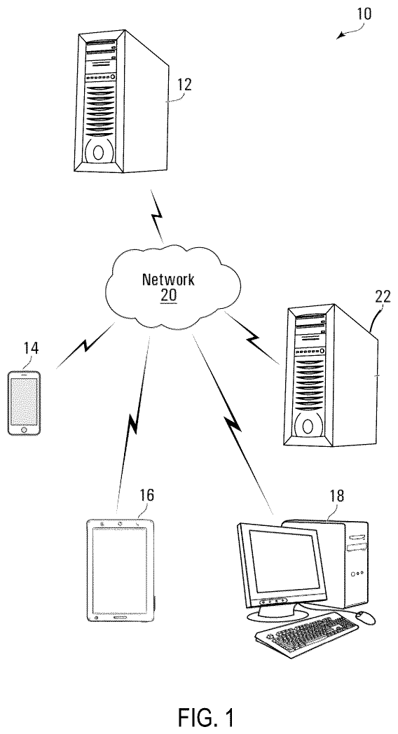

Referring now to FIG. 1, shown therein is an example computer network system 10 for sentiment analysis of patient (or other user) data, according to an embodiment.

The system 10 includes a server platform 12 which receives data from a plurality of sources 14, 16, and 18. The sources may include, for example, receiving data from a mobile phone 14, a tablet or other portable computing device 16, or a desktop computing device 18. The sources 14, 16, and 18, may be used by a patient, a healthcare professional, an employee of a pharmaceutical company, etc. That is, patient data may be collected directly from a patient or may be provided to the server platform 12, indirectly via someone who interacted with the patient. The server platform 12 also communicates with a patient data database 22, which stores patient data from the other sources.

The server platform 12 may be a purpose-built machine designed specifically for receiving, storing, and processing patient data where such access, storage, and processing of the data is authorized and follows all required policies and regulations. The server platform 12 employs artificial intelligence algorithms, for example machine learning or natural language processing algorithms, to analyze a sentiment of the patient data for a specific sentiment target. For example, the server platform 12 may analyze patient data from a cohort of patients to determine how a pharmaceutical is being tolerated, or, as another example, the server platform 12 may analyze patient data from a single patient to determine a mental health status of the patient.

The server platform 12, sources 14, 16, and 18 and patient data database 22 may be a server computer, desktop computer, notebook computer, tablet, PDA, smartphone, or another computing device. The devices 12, 14, 16, 18, 22 may include a connection with the network 20 such as a wired or wireless connection to the Internet. In some cases, the network 20 may include other types of computer or telecommunication networks. The devices 12, 14, 16, 18, 22 may include one or more of a memory, a secondary storage device, a processor, an input device, a display device, and an output device. Memory may include random access memory (RAM) or similar types of memory. Also, memory may store one or more applications for execution by processor. Applications may correspond with software modules comprising computer executable instructions to perform processing for the functions described below. Secondary storage device may include a hard disk drive, floppy disk drive, CD drive, DVD drive, Blu-ray drive, or other types of non-volatile data storage. Processor may execute applications, computer readable instructions or programs. The applications, computer readable instructions or programs may be stored in memory or in secondary storage or may be received from the Internet or other network 20. Input devices may include any device for entering information into device 12, 14, 16, 18, 22. For example, input device may be a keyboard, keypad, cursor-control device, touchscreen, camera, or microphone. Display devices may include any type of device for presenting visual information. For example, display devices may be a computer monitor, a flat-screen display, a projector, or a display panel. Output device may include any type of device for presenting a hard copy of information, such as a printer for example. Output devices may also include other types of output devices such as speakers, for example. In some cases, device 12, 14, 16, 18, 22 may include multiple of any one or more of processors, applications, software modules, second storage devices, network connections, input devices, output devices, and display devices.

Although devices 12, 14, 16, 18, 22 are described with various components, one skilled in the art will appreciate that the devices 12, 14, 16, 18, 22 may in some cases contain fewer, additional, or different components. In addition, although aspects of an implementation of the devices 12, 14, 16, 18, 22 may be described as being stored in memory, one skilled in the art will appreciate that these aspects can also be stored on or read from other types of computer program products or computer-readable media, such as secondary storage devices, including hard disks, floppy disks, CDs, or DVDs; a carrier wave from the Internet or other network; or other forms of RAM or ROM. The computer-readable media may include instructions for controlling the devices 12, 14, 16, 18, 22 and/or processor to perform a particular method.

Server platform 12 may be configured to receive a plurality of information, from each of the devices 14, 16, 18, and 22. Generally, the information may comprise at least an identifier identifying the patient (or other user). For example, the information may comprise one or more of a username, e-mail address, password, or social media handle.

In response to receiving information, the server platform 12 may store the information in storage database. The storage may correspond with secondary storage of the patient data database 22. Generally, the storage database may be any suitable storage device such as a hard disk drive, a solid state drive, a memory card, or a disk (e.g. CD, DVD, or Blu-ray etc.). Also, the storage database may be locally connected with server platform 12. In some cases, the storage database may be located remotely from server platform 12 and accessible to server platform 12 across a network for example. In some cases, storage databases may comprise one or more storage devices located at a networked cloud storage provider.

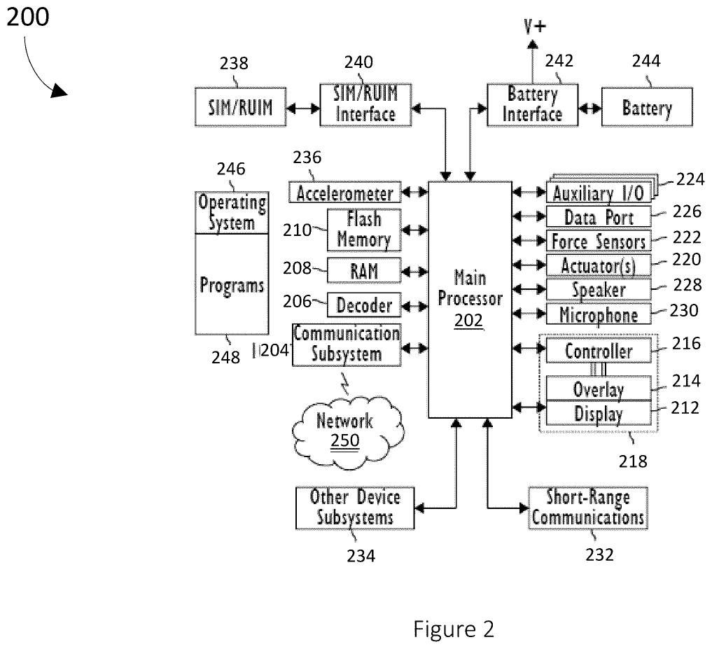

Referring now to FIG. 2, shown therein is a block diagram of a computing device 200 of the system 10 of FIG. 1, according to an embodiment. The computing device 200 may be, for example, any one of devices 12, 14, 16, 18, 22 of FIG. 1.

The computing device 200 includes multiple components such as a processor 202 that controls the operations of the computing device 200. Communication functions, including data communications, voice communications, or both may be performed through a communication subsystem 204. Data received by the computing device 200 may be decompressed and decrypted by a decoder 206. The communication subsystem 204 may receive messages from and send messages to a wireless network 250.

The wireless network 250 may be any type of wireless network, including, but not limited to, data-centric wireless networks, voice-centric wireless networks, and dual-mode networks that support both voice and data communications.

The computing device 200 may be a battery-powered device and as shown includes a battery interface 242 for receiving one or more rechargeable batteries 244.

The processor 202 also interacts with additional subsystems such as a Random Access Memory (RAM) 208, a flash memory 210, a display 212 (e.g. with a touch-sensitive overlay 214 connected to an electronic controller 216 that together comprise a touch-sensitive display 218), an actuator assembly 220, one or more optional force sensors 222, an auxiliary input/output (I/O) subsystem 224, a data port 226, a speaker 228, a microphone 230, short-range communications systems 232 and other device subsystems 234.

In some embodiments, user-interaction with the graphical user interface may be performed through the touch-sensitive overlay 214. The processor 202 may interact with the touch-sensitive overlay 214 via the electronic controller 216. Information, such as text, characters, symbols, images, icons, and other items that may be displayed or rendered on a computing device generated by the processor 202 may be displayed on the touch-sensitive display 218.

The processor 202 may also interact with an accelerometer 236 as shown in FIG. 2. The accelerometer 236 may be utilized for detecting direction of gravitational forces or gravity-induced reaction forces.

To identify a subscriber for network access according to the present embodiment, the computing device 200 may use a Subscriber Identity Module or a Removable User Identity Module (SIM/RUIM) card 238 inserted into a SIM/RUIM interface 240 for communication with a network (such as the wireless network 250). Alternatively, user identification information may be programmed into the flash memory 210 or performed using other techniques.

The computing device 200 also includes an operating system 246 and software components 248 that are executed by the processor 202 and which may be stored in a persistent data storage device such as the flash memory 210. Additional applications may be loaded onto the computing device 200 through the wireless network 250, the auxiliary I/O subsystem 224, the data port 226, the short-range communications subsystem 232, or any other suitable device subsystem 234.

In use, a received signal such as a text message, an e-mail message, web page download, or other data may be processed by the communication subsystem 204 and input to the processor 202. The processor 202 then processes the received signal for output to the display 212 or alternatively to the auxiliary I/O subsystem 224. A subscriber may also compose data items, such as e-mail messages, for example, which may be transmitted over the wireless network 250 through the communication subsystem 204.

For voice communications, the overall operation of the computing device 200 may be similar. The speaker 228 may output audible information converted from electrical signals, and the microphone 230 may convert audible information into electrical signals for processing.

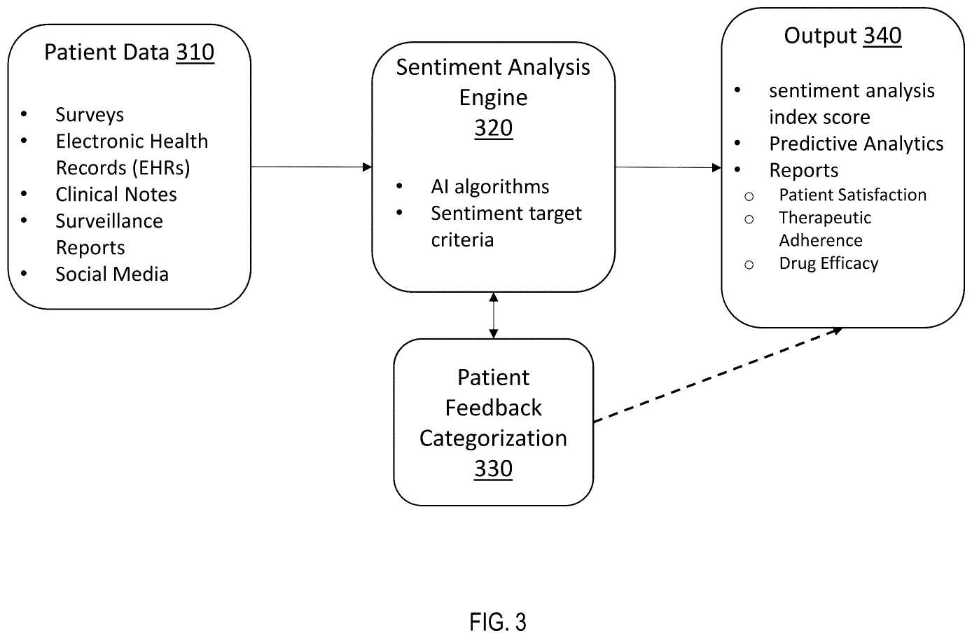

FIG. 3 is a block diagram showing the flow of data through a sentiment analysis system, according to an embodiment. Patient data 310 is received by the sentiment analysis engine 320 which analyzes the patient data 310 using at least one artificial intelligence algorithm trained to recognize sentiment. The sentiment analysis engine generates at least one output 340 provided insights of the sentiment of the patient data.

The patient data 310 may be received directly from the patient (i.e., the patient is directly providing information to the sentiment analysis system) or indirectly through a healthcare professional or other service which provides the data to the sentiment analysis engine 320.

Examples of patient data 310 include surveys, electronic health records (EHRs), clinical notes, surveillance reports, and social media.

Surveys may be provided to the patient by a healthcare professional or a service such as a mobile application. The survey(s) may be related to a specific therapy or trial the patient is a part of, e.g., the patient is participating in a clinical trial and completes surveys at regular intervals.

Electronic health records may include any health records for the patient which are created or stored electronically. The health records may be created by the patient or by a healthcare professional or other related employee.

Clinical notes may include any notes generated by a doctor or other healthcare practitioner. The notes may be digitized. For clinical notes, the sentiment analyzed by the sentiment analysis engine 320 may be that of the healthcare practitioner and not the patient, but may still be taken as indirect evidence of the sentiment of a patient.

A surveillance report may be provided by the patient or by a healthcare professional.

Access to data from social media may be provided with the permission of the patient. The sentiment if the patient may be analyzed based on statements or information provided by the patient on their social media regarding their health and wellbeing.

The provided types of patient data 310 are limited examples and other types of patient data 310 may be received by the sentiment analysis engine 320.

In some situations, the data may be de-personalized to remove any identifying information from the process of sentiment analysis. De-personalization may include removing identifying information of a patient(s).

In other situations, non-patient sensitive information may be removed or blocked during the sentiment analysis. For example, the names of certain drugs or illnesses may be removed from the data.

While the term “patient data” is used, the data received by the sentiment analysis engine may not be from a patient who is currently working with a healthcare practitioner(s) but rather from a user of a healthcare application or educational application. That is, data may be provided by any person regardless of their status as an official patient in any capacity.

Patient data 310 may be from a single patient or from a cohort of patients.

The sentiment analysis engine 320 includes artificial intelligence (AI) algorithms which have been trained to analyze data to determine sentiment. The AI algorithms may include machine learning algorithms and/or natural language processing algorithms. The AI algorithms may be trained and refined for specific sentiment targets, e.g., finding adverse drug reactions, determining a mental health status of a patient, etc.

The AI algorithms may be trained by supervised and/or unsupervised training with diverse data sets from multiple different sources and source types. Some of the AI algorithms may be trained to identify which parts of the data are important for sentiment analysis, while other AI algorithms may be trained to identify sentiment from the important data. Some AI algorithms may incorporate both functionalities and/or more functionalities. Important data may include, for example, notes or comments on the physical or mental state of the patient, while unimportant data would include, for example, the patient's phone number or address.

The training data sets may include open source data sets, with both testing and training data sets used to train and refine algorithms.

As an example, training data may include health care data from a patient wherein it is known what the sentiment or attitude of the patient was (as scored by non-AI algorithm tools, e.g., surveys, healthcare provider opinions, etc.) at particular points throughout a treatment plan.

For a given sentiment analysis task multiple algorithms and multiple series of algorithms may be used.

The sentiment analysis engine 320 may receive, as input, sentiment target criteria which enables the sentiment analysis engine to select a specific AI algorithm for a sentiment analysis task. A user of the sentiment analysis engine 320 may input the sentiment target criteria based on the task, e.g., pharmacovigilance, academic research, insurance claims, etc. The sentiment analysis engine 320 may employ more than one AI algorithm for a given sentiment analysis task, wherein the sentiment analysis engine 320 considers the level of consensus between AI algorithms when generating any outputs. The sentiment analysis engine may output a confidence score based on the consensus.

The sentiment analysis engine 320 analyzes the patient data 310 for sentiment regarding a sentiment target using the selected AI algorithm tools and generates at least one output 340. Before the output can be generated the sentiment analysis engine 320 may categorize the patient data by patient feedback categorization 330.

The sentiment analysis engine 320 includes algorithms or sub-algorithms for patient feedback categorization 330. The step of patient feedback categorization may occur before the patient data is input into the AI algorithms for sentiment analysis or may be a function performed as part of the AI algorithms for sentiment analysis. Patient feedback categorization 330 may categorize feedback into actionable categories or key themes. For example, actionable categories may include treatment satisfaction, service experience, or side-effect concerns. The actionable categories may each be a sentiment target or may be included within a sentiment target. For example, the treatment satisfaction, service experience, and side-effect concerns may all be included in a sentiment target of patient adherence.

Patient feedback categorization 330 information may be provided as an output of the sentiment analysis engine 320. That is, a user may be able to see which data has been sorted into which category, or the amount of data present for each category.

The outputs 340 may include a sentiment analysis index score for the sentiment target(s), wherein the score represents a positive, neutral, or negative sentiment, and may represent a degree of sentiment within the categories of positive, neutral, and negative. Where there are multiple sentiment targets, each target may receive a separate sentiment analysis index score.

Where the patient data is for a single patient, the sentiment analysis index score represents the sentiment of the patient. Where the patient data is from a cohort of patients, the sentiment analysis index score may represent an overall sentiment of the entire cohort. The sentiment analysis index score may also be presented as a range of scores for the patients within the cohort. The sentiment analysis index score may also be refinable based on characteristics of the patients in the patient cohort. That is, a user of the sentiment analysis system may be able to view sentiment analysis index scores by demographics such as age of patient, gender of patient, ethnicity of patient, etc.

There may be multiple sentiment targets for a given task or set of patient data. For example, for pharmacovigilance, the sentiment analysis engine 320 may be tasked with finding sentiment associated with adverse drug reactions, sentiment associated with patient adherence, and sentiment associated with drug efficacy.

The outputs 340 may also include predictive analytics, for example the likelihood of continued patient adherence.

The outputs 340 may include reports, for example, for patient satisfaction, therapeutic adherence, and drug efficacy.

The outputs are provided to decision-makers or other stakeholders who are able to effect a change based on the results shown in the outputs. For example, if adverse drug reactions are identified, the facilitator of a clinical trial may pause or shut down the trial. As another example, if a sentiment analysis shows that a patient is having mental health difficulties, a healthcare provider for the patient may intervene.

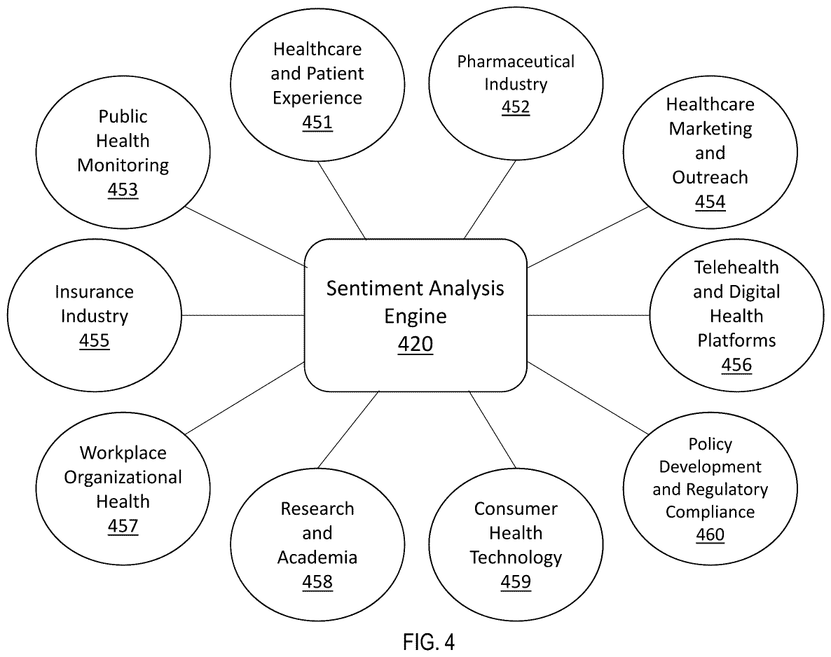

FIG. 4 is a block diagram showing the possible applications of a sentiment analysis system with a sentiment analysis engine 420. The applications include healthcare and patient experience 451, pharmaceutical industry 452, public health monitoring 453, healthcare marketing and outreach 454, insurance industry 455, telehealth and digital health platforms 456, workplace organizational health 457, research and academia 458, consumer health technologies 459, and policy development and regulatory compliance 460.

The healthcare and patient experience application 451 may include: i) monitoring patient satisfaction, by analyzing patient feedback from surveys, reviews, and portals to improve healthcare services, wherein the patient feedback may be regarding patient experience in hospitals, clinics, and pharmaceutical services ii) providing therapeutic adherence support by identifying patient challenges or dissatisfaction with prescribed treatments to guide personalized interventions and iii) providing mental health assessment by detecting signs of stress, anxiety, or depression in patient communication for timely support. In this application, real-time insights may be provided to healthcare teams to improve service quality and care delivery.

The pharmaceutical industry application 452 may include: i) performing pharmacovigilance whereby drug efficacy, and adverse drug reactions (ADRs) and medication safety issues are monitored by analyzing real-world patient feedback, ii) generating drug development feedback, by evaluating patient sentiment in clinical trial reports to enhance drug design and trial protocols, and iii) performing post-marketing surveillance by analyzing consumer reviews and social media discussions to assess drug performance and compliance.

The public health monitoring application 453 may include: i) providing epidemiological insights by detecting trends in patient discussions to identify emerging health issues or outbreaks, and ii) analyzing health campaign effectiveness by measuring a public response to health awareness campaigns and policies.

The healthcare marketing and outreach application 454 may include: i) providing brand sentiment analysis: understanding public perception of healthcare services, pharmaceutical products, or health initiatives, and ii) enabling targeted campaigns by tailoring communication strategies based on audience sentiment and engagement trends.

The insurance industry application 455 may include: i) providing claims sentiment analysis by analyzing customer sentiment in claim submissions and interactions to improve service quality, and ii) providing fraud detection by identifying anomalies or inconsistencies in claim narratives using sentiment patterns.

The telehealth and digital health platforms application 456 may include: i) providing virtual consultation enhancement by analyzing patient sentiment during teleconsultations to improve engagement and care delivery, and ii) improving ai-powered chatbots by enhancing chatbot interactions by incorporating sentiment-aware responses for better patient support.

The workplace and organizational health application 457 may include: i) providing employee wellness monitoring by assessing sentiment in employee health feedback to guide wellness initiatives, and ii) providing an organizational sentiment by monitoring overall sentiment within a healthcare organization to foster a positive work environment.

The research and academia application 458 may include: i) enabling patient-reported outcome studies by using sentiment analysis to complement quantitative metrics in patient outcome research, and ii) providing a health literacy assessment: by evaluating the clarity and effectiveness of health education materials based on audience sentiment.

The consumer health technologies application 459 may include: i) providing health app feedback by monitoring user sentiment regarding features, usability, and effectiveness of health-focused mobile applications, and ii) providing wearables integration: analyzing sentiment data captured through wearable devices for personalized health insights.

The policy development and regulatory compliance application 460 may include: i) providing regulatory monitoring by identifying and addressing compliance issues from public and stakeholder feedback, and ii) providing policy impact analysis by measuring public sentiment towards new health regulations or reforms to refine policies.

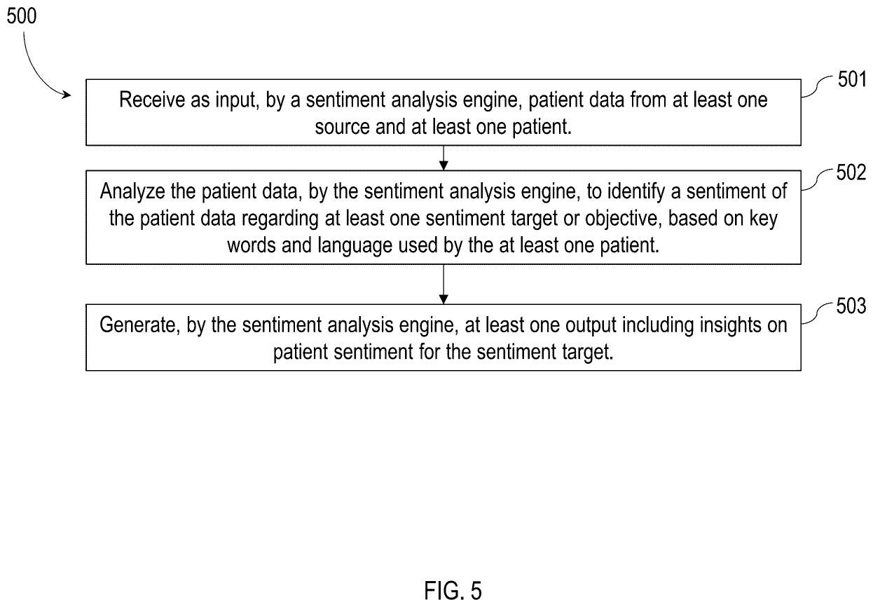

FIG. 5 is a flow diagram of a method 500 of using a sentiment analysis engine to provide insights regarding patient data.

At 501, the sentiment analysis engine receives as input patient data from at least one source and at least one patient. The source of the patient data is the place where the patient data is generated and/or stored. The sources may be surveys, EHRs, clinical notes, social media, interviews, or any other form of generating and storing information provided directly or indirectly by a patient.

In some embodiments, the “patient” may be a user of a system and may not be receiving medical care.

In some embodiments, the at least one patient is a single patient (or user) and sentiment is analyzed for only the single patient to provide information about the individual patient. In other embodiments, the at least one patient is a group or cohort of patients (or users) wherein the patient data is analyzed for an overall sentiment of the cohort or to determine how sentiment differs between sub-populations of the cohort.

When the data is received and accessed by the sentiment analysis system, all relevant protocols and standards for patient data are followed.

At 502, a sentiment analysis engine analyzes the patient data to identify at a sentiment for at least one sentiment target (or objective), based on keywords and language used by or about the at least one patient in the patient data. Examples of sentiment targets include pharmaceuticals, therapies, educational materials, virtual assistants, etc. The sentiment which the patient(s) have towards the target can be used to predict outcomes or suggest improvements related to the sentiment targets.

The sentiment analysis engine includes at least one AI model (or algorithm) trained to analyze data and generate insights regarding at least one sentiment of the data. The AI algorithms may include machine learning algorithms and natural language processing algorithms. Other types of AI algorithms may also be used. The algorithms are trained using training data for which sentiments have already been determined and which represents a wide variety of sources and patients.

The AI models scan through the patient data for key words and phrases, density of words, usage of words, frequency of words, and overall positive, negative, or neutral views to determine sentiment(s). The AI models may combine data generated based on the key words and phrases with demographic data points for each patient (e.g. age, gender, etc.) that may contribute to the sentiment towards the sentiment target.

The sentiment analysis engine may determine which algorithm(s) to use to analyze the patient data based on sentiment target criteria input by a user for the specific sentiment target of interest to the user.

The sentiment algorithm may use a single algorithm for a sentiment target or may use multiple algorithms for a sentiment target. When multiple algorithms are used for a single sentiment target each algorithm may generate its own insights and/or insights may be generated based on a consensus of multiple algorithms.

At 503, the sentiment analysis engine provides at least one output regarding patient (or user) sentiment for the sentiment target(s).

The at least one ouput may include a sentiment analysis index score or multiple sentiment analysis index scores for each sentiment target. Other outputs may include predictive analysis, e.g., likelihood of patient adherence, and reports about the sentiment target including current outcomes as well as suggestions for the future. For example, a report may include a current level of patient satisfaction and provide suggestions to increase patient satisfaction.

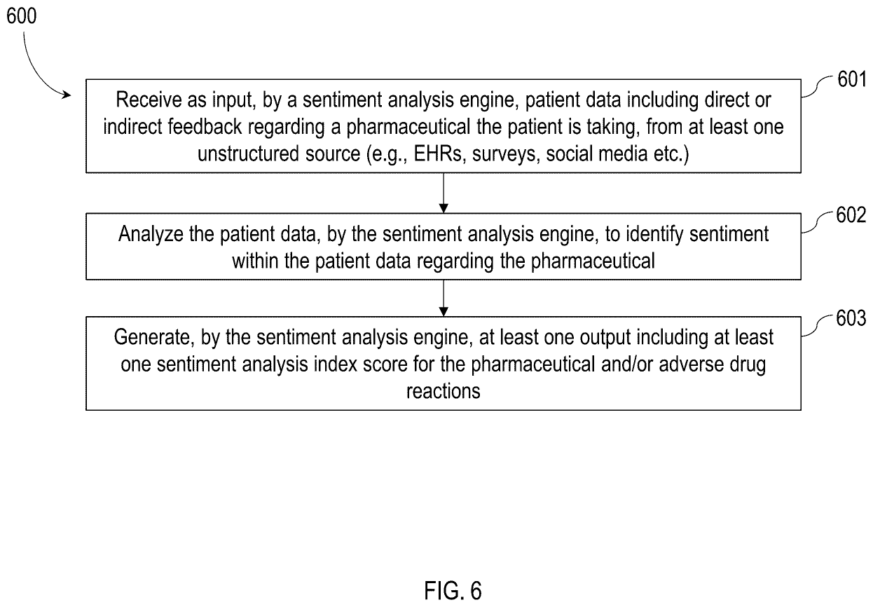

FIGS. 6-15 are flow diagrams each showing a specific application of the method 500 and sentiment analysis engine described for FIG. 5. The applications of FIG. 6-15 discuss the specific type of patient data used for a specific sentiment target and the likely output for said sentiment target. Each of the methods of FIGS. 6-15 follows the basic method of FIG. 5, and, therefore, the basic method is not repeated in its entirely for each method.

In most applications, the patient data represents hundreds or more patients wherein a sentiment toward a sentiment target is analyzed over the entire cohort. However, in some situations patient data is analyzed by the sentiment analysis engine for a single patient and compared to historical sentiment data for the sentiment target in question.

FIG. 6 is a flow diagram of a method 600 for a sentiment-based pharmacovigilance system using a sentiment analysis engine to analyze a sentiment of pharmacovigilance patient data, to provide an output of a sentiment analysis index score and/or newly identified adverse drug reactions.

At 601, the sentiment analysis engine receives patient data including direct (from the patient) or indirect (from a healthcare provider, social media, etc.) feedback for a patient taking a specific pharmaceutical, from at least on unstructured source (e.g., EHRs, surveys, social media posts, etc.). The patient data for the pharmacovigilance system may be received and analyzed in real-time and on an ongoing basis.

The patient data may be from a single patient or from a cohort of patients.

At 602, the sentiment analysis engine analyzes the patient data to identify sentiment regarding the pharmaceutical being monitored.

At 603, the sentiment analysis engine generates at least one output. The output may include a sentiment analysis index score which indicates a patient sentiment about the pharmaceutical. Sentiment analysis index scores may be qualitative, i.e., positive, negative, neutral, or quantitative, i.e., a scale of 1-10 where 1 is poor and 10 is excellent.

Sentiment analysis index scores may be for a single patient or a cohort of patients.

Other outputs may include identification of adverse drug reactions, predictions of patient adherence, and/or patient impressions of drug efficacy. For example, unique AI algorithms could be used to identify safety signals within the patient data that identify the adverse drug reactions.

Outputs for an individual patient may include the likelihood of patient adherence. Outputs for a cohort of patients may include overall drug efficacy.

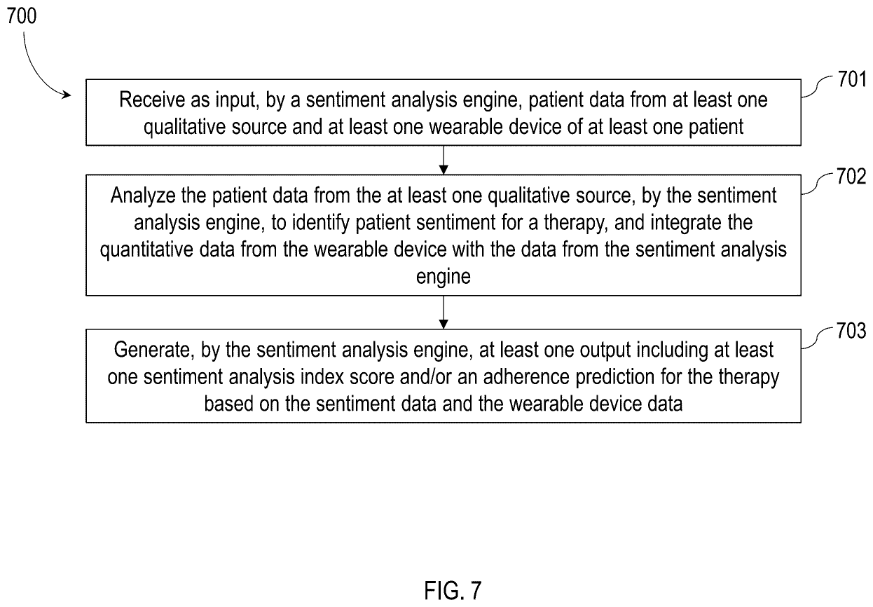

FIG. 7 is a flow diagram of a method 700 for predictive patient adherence using a sentiment analysis engine to analyze a sentiment of patient data together with wearable device data, to provide an output of a sentiment analysis index score and/or a prediction of adherence to a therapy.

At 701, the sentiment analysis engine receives patient data from at least one qualitative source (e.g., surveys, clinical notes, social media, etc.) and at least one wearable device. The patient data for the predictive patient adherence system may be received and analyzed in real-time and on an ongoing basis.

The patient data is most likely from a single patient but may be from a cohort of patients.

At 702, the sentiment analysis engine analyzes the patient data to identify sentiment regarding a therapy the patient is undertaking and integrates the quantitative data from the at least one wearable device with the sentiment analysis data.

At 703, the sentiment analysis engine generates at least one output. The output may include a sentiment analysis index score which indicates a patient sentiment about the therapy. Sentiment analysis index scores may be qualitative, i.e., positive, negative, neutral, or quantitative, i.e., a scale of 1-10 where 1 is poor and 10 is excellent.

Sentiment analysis index scores may be for a single patient or a cohort of patients.

Other outputs may include a prediction of patient adherence, either individually or for a cohort. For example, the sentiment analysis engine may output a risk score for non-adherence by combining sentiment data with physical activity and/or biometrics.

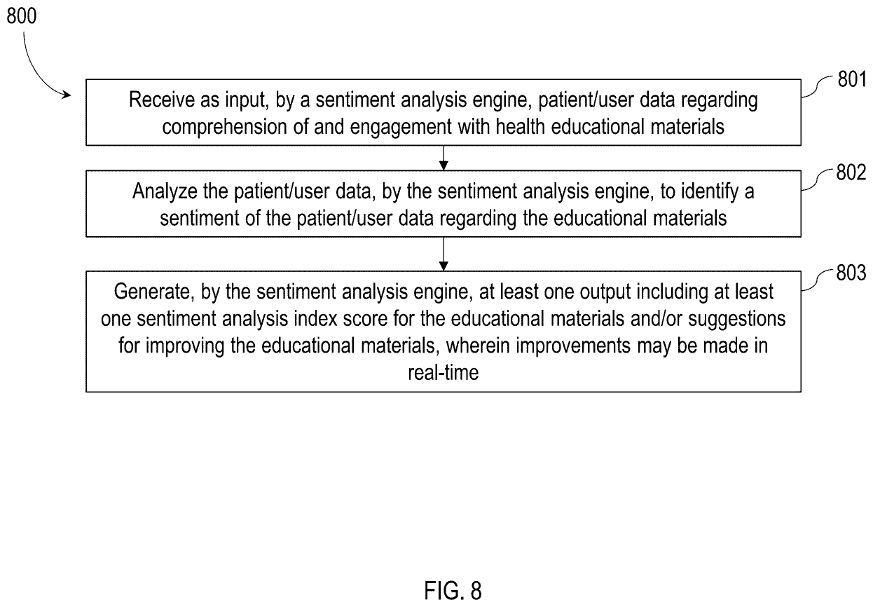

FIG. 8 is a flow diagram of a method 800 for a dynamic sentiment-based health literacy tool using a sentiment analysis engine to analyze a sentiment of patient/user data regarding health educational material, to provide an output of a sentiment analysis index score and/or suggestions for improving the educational materials.

At 801, the sentiment analysis engine receives patient/user data regarding comprehension of and engagement with health educational materials.

The patient/user data may be from a single patient and/or from a cohort of patients.

At 802, the sentiment analysis engine analyzes the patient data to identify sentiment regarding the educational materials.

At 803, the sentiment analysis engine generates at least one output. The output may include a sentiment analysis index score which indicates a patient/user sentiment about the educational materials. Sentiment analysis index scores may be qualitative, i.e., positive, negative, neutral, or quantitative, i.e., a scale of 1-10 where 1 is poor and 10 is excellent.

Other outputs may include suggestions for improving the educational materials. Improvements may be adopted in real-time while a user is using the educational materials to facilitate better learning by the user. Improvements may be adopted for all educational materials based on cohort data.

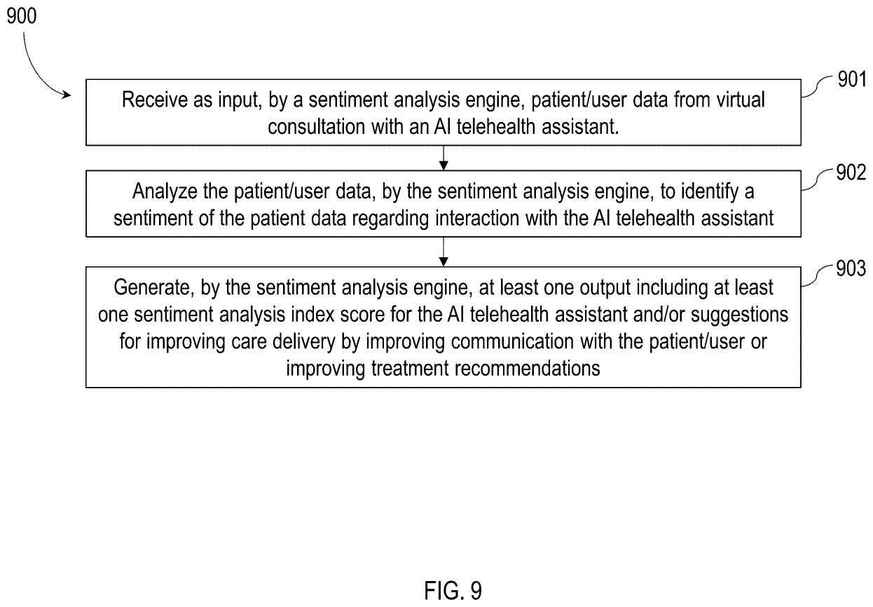

FIG. 9 is a flow diagram of a method 900 for a virtual sentiment-aware telehealth assistant using a sentiment analysis engine to analyze a sentiment of patient/user data generated during interaction with an AI telehealth assistant, to provide an output of a sentiment analysis index score and/or suggestions for improving the function of the AI telehealth assistant.

At 901, the sentiment analysis engine receives patient/user data from a virtual consultation with an AI telehealth assistant. The patient data from interacting with the AI telehealth assistant may be received and analyzed in real-time.

The patient data is most likely from a single patient to update the assistant for the patient but may be from a cohort of patients to make changes to the assistant for all users.

At 902, the sentiment analysis engine analyzes the patient data to identify sentiment of the patient regarding interaction with the AI telehealth assistant.

At 903, the sentiment analysis engine generates at least one output. The output may include a sentiment analysis index score which indicates a patient sentiment about the AI telehealth assistant. Sentiment analysis index scores may be qualitative, i.e., positive, negative, neutral, or quantitative, i.e., a scale of 1-10 where 1 is poor and 10 is excellent.

Other outputs may include suggestions for improving the delivery of care to the patient by improving communication with the patient and/or improving treatment recommendations which results from the consultation. This enables more effective health care delivery in terms of both time spent by the patient and healthcare providers, as well as decreasing the time to achieve effective treatment/therapies.

Data from a single patient/user may be used to improve that patient/user's experience, while data from a cohort of users may be used to improve the AI telehealth assistant's overall performance.

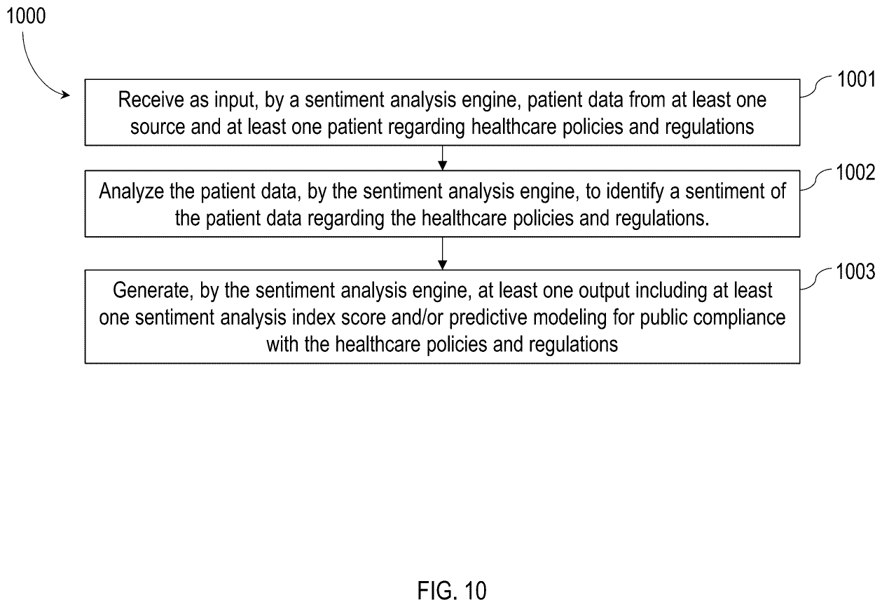

FIG. 10 is a flow diagram of a method 1000 for real-time regulatory feedback monitoring using a sentiment analysis engine to analyze a sentiment of patient (or members of the public) data regarding healthcare policies and regulations (e.g., vaccine schedules), to provide an output of a sentiment analysis index score and/or a predictive model for public compliance with the healthcare policies and/or regulations.

At 1001, the sentiment analysis engine receives patient data from at least one source and at least one patient. The patient data may be received and analyzed in real-time and on an ongoing basis.

The patient data may be from a single patient or a cohort of patients.

At 1002, the sentiment analysis engine analyzes the patient data to identify sentiment regarding the healthcare policies and regulations.

At 1003, the sentiment analysis engine generates at least one output. The output may include a sentiment analysis index score which indicates a patient sentiment about the healthcare policies and regulations. Sentiment analysis index scores may be qualitative, i.e., positive, negative, neutral, or quantitative, i.e., a scale of 1-10 where 1 is poor and 10 is excellent.

Other outputs may include predictive modelling for public compliance with the healthcare policies and regulations. Predictive modelling at a high level allows for more effective policymaking.

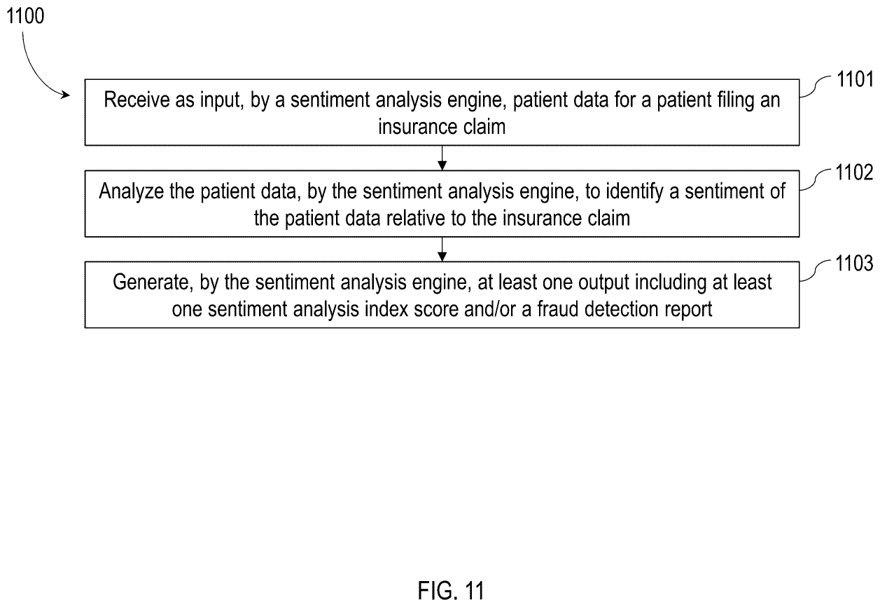

FIG. 11 is a flow diagram of a method 1100 for sentiment-guided fraud detection for insurance claims using a sentiment analysis engine to analyze a sentiment of a patient filing an insurance claim, to provide an output of a sentiment analysis index score and/or a fraud detection report.

At 1101, the sentiment analysis engine receives patient data for a patient filing an insurance claim.

At 1102, the sentiment analysis engine analyzes the patient data to identify a sentiment of the patient relative to the insurance claim.

At 1103, the sentiment analysis engine generates at least one output. The output may include a sentiment analysis index score which indicates a patient sentiment about the insurance claim. Sentiment analysis index scores may be qualitative, i.e., positive, negative, neutral, or quantitative, i.e., a scale of 1-10 where 1 is poor and 10 is excellent.

Other outputs may include a fraud detection report which indicates the likelihood that the patient is committing fraud.

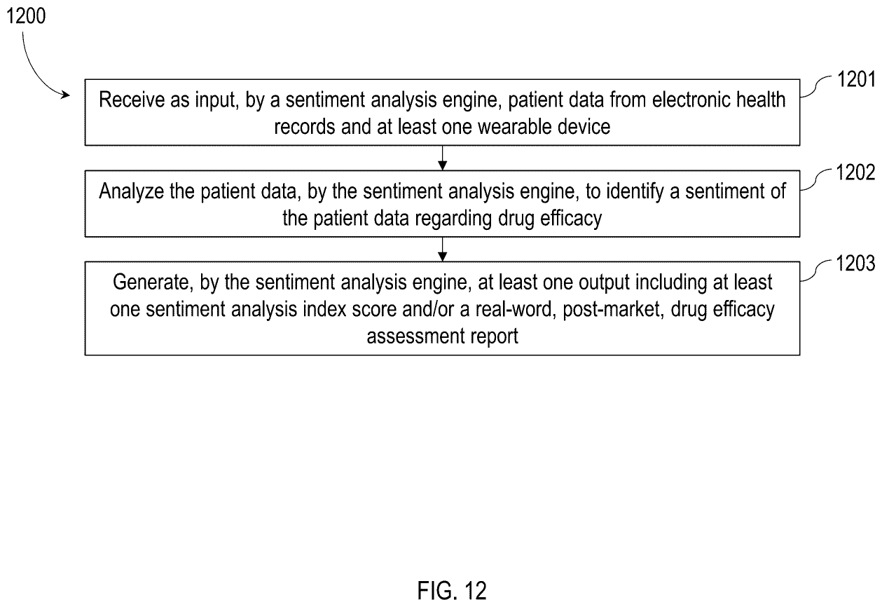

FIG. 12 is a flow diagram of a method 1200 multi-modal real-world evidence analysis using a sentiment analysis engine to analyze a sentiment of patient data from electronic health records and at least one wearable device, wherein the patient has been prescribed a pharmaceutical, and providing an output of a sentiment analysis index score and/or a drug efficacy report.

At 1201, the sentiment analysis engine receives patient data from EHRs and at least one wearable device. The patient data may be received and analyzed in real-time and on an ongoing basis.

The patient data may be from a single patient or may be from a cohort of patients.

At 1202, the sentiment analysis engine analyzes the patient data to identify sentiment regarding a drug the patient is taking and integrates the quantitative data from the at least one wearable device with the sentiment analysis data.

At 1203, the sentiment analysis engine generates at least one output. The output may include a sentiment analysis index score which indicates a patient sentiment about the efficacy of the drug. Sentiment analysis index scores may be qualitative, i.e., positive, negative, neutral, or quantitative, i.e., a scale of 1-10 where 1 is poor and 10 is excellent.

Sentiment analysis index scores may be for a single patient or a cohort of patients.

Other outputs may include an assessment report indicating the efficacy of the drug in the real-world, post-market. This enables ongoing follow-up study for post-market drug assessment that would otherwise be logistically difficult as well as cost and time prohibitive.

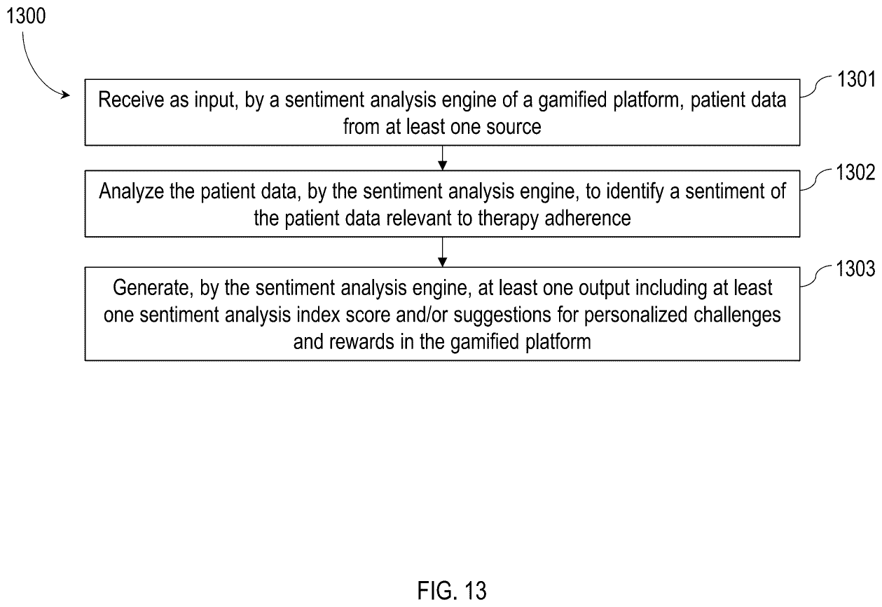

FIG. 13 is a flow diagram of a method 1300 of using a sentiment analysis engine to analyze a sentiment of patient data related to a gamified health platform for a patient prescribed a specific therapy, to provide an output of a sentiment analysis index score and/or suggestions for personalized challenges and rewards on the gamified platform. The challenges and rewards are used to motivate the patient to follow a therapeutic plan.

At 1301, the sentiment analysis engine receives patient data from a gamified health and/or wellness platform. The patient data may be received and analyzed in real-time and on an ongoing basis.

The patient data may be from a single patient or a cohort of patients.

At 1302, the sentiment analysis engine analyzes the patient data to identify a sentiment of the patient relative to therapies suggested by the gamified platform.

At 1303, the sentiment analysis engine generates at least one output. The output may include a sentiment analysis index score which indicates a patient sentiment about the therapy suggested or prescribed by the gamified platform or the gamified platform itself. Sentiment analysis index scores may be qualitative, i.e., positive, negative, neutral, or quantitative, i.e., a scale of 1-10 where 1 is poor and 10 is excellent.

Other outputs may include suggestions for personalized challenges and rewards in the gamified platform which may improve patient adherence to the suggested therapies. This allows for the integration of behavioral science with AI healthcare tools.

Data from an individual patient/user may be used to improve the patient/user's adherence, while data from a cohort of patients/users may be used to improve the overall performance of the gamified platform.

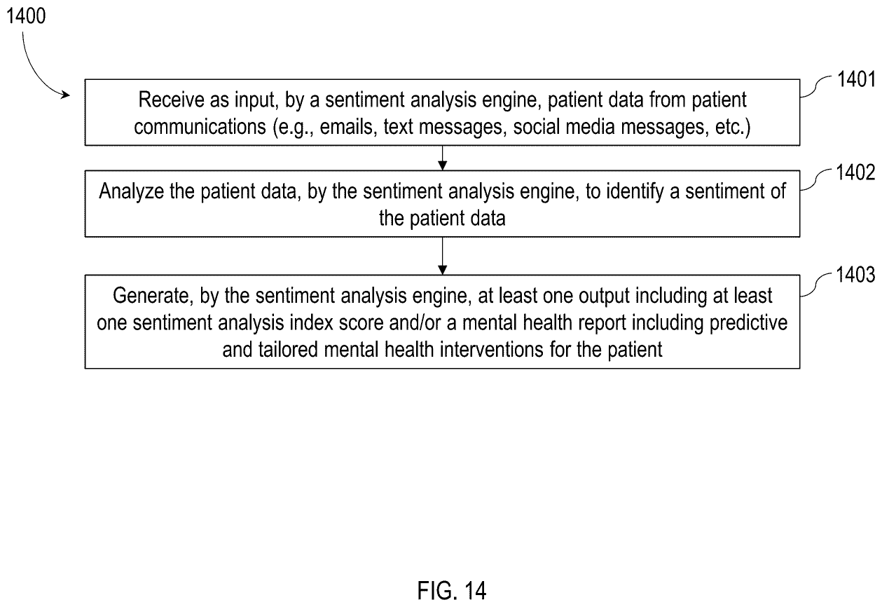

FIG. 14 is a flow diagram of a method 1400 for AI-driven mental health risk detection using a sentiment analysis engine to analyze a sentiment of patient data, to provide an output of a sentiment analysis index score and/or a mental health report for the patient.

At 1401, the sentiment analysis engine receives patient data from patient communications including emails, text messages, and/or social media messages. The patient data may be received and analyzed in real-time and on an ongoing basis.

At 1402, the sentiment analysis engine analyzes the patient data to identify sentiment regarding or related to the mental health of the patient.

At 1403, the sentiment analysis engine generates at least one output. The output may include a sentiment analysis index score which indicates the mental health of the patient. Sentiment analysis index scores may be qualitative, i.e., positive, negative, neutral, or quantitative, i.e., a scale of 1-10 where 1 is poor and 10 is excellent.

Other outputs may include a mental health report including predictive and tailored mental health interventions for the patient. This allows for the integration of personalized sentiment analysis with mental health care.

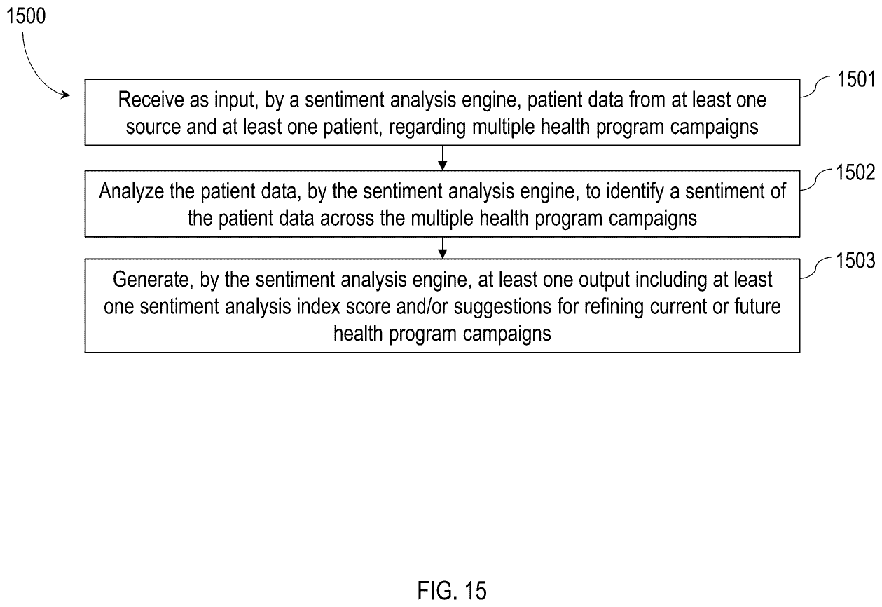

FIG. 15 is a flow diagram of a method 1500 for sentiment-based competitive health campaign analysis using a sentiment analysis engine to analyze a sentiment of patient data for multiple health campaigns, to provide an output of a sentiment analysis index score and/or suggestions for refining a current health campaign or designing future health campaigns.

At 1501, the sentiment analysis engine receives patient data from at least one source and at least one patient regarding multiple health program campaigns.

At 1502, the sentiment analysis engine analyzes the patient data to identify sentiment of the patient data across each of the multiple health program campaigns.

At 1503, the sentiment analysis engine generates at least one output. The output may include sentiment analysis index scores for each campaign which indicate patient sentiment about each campaign. Sentiment analysis index scores may be qualitative, i.e., positive, negative, neutral, or quantitative, i.e., a scale of 1-10 where 1 is poor and 10 is excellent.

Other outputs may include suggestions for refining current and/or future health program campaigns. This enables stakeholders to benchmark and refine campaigns, particularly in competitive healthcare markets or to provide a unique campaign.

For all of the embodiments and applications of the sentiment analysis system described herein, the actual outcomes regarding the sentiment target may be tracked and then used along with the associated patient data to continue training the AI algorithms used by the sentiment analysis engine. For example, patient adherence may be tracked for pharmacovigilance to determine how accurately the sentiment analysis engine predicted adherence, and the actual patient adherence outcomes may be associated with the patient data and used to train a patient adherence AI algorithm to predict adherence more accurately in the future.

In embodiments, where individual patient data is used to provide outputs for individual patients, the patient data from multiple patients, and the actual outcomes following sentiment analysis, may be aggregated and used to train or refine the AI algorithms used by the sentiment analysis engine.

While the above description provides examples of one or more apparatus, methods, or systems, it will be appreciated that other apparatuses, methods, or systems may be within the scope of the claims as interpreted by one of skill in the art.

1. A computer system for analyzing a sentiment of user data, the system comprising:
at least one processor configured to execute processor-executable instructions stored on at least one non-transitory memory to implement:
a sentiment analysis engine configured to:
receive user data from at least one source and at least user;
analyze the user data to identify a sentiment of the user data, for at least one sentiment target, based on keywords and language found within the user data, using at least one artificial intelligence (AI) model; and
generate at least one output indicating the sentiment of the user data for the at least one sentiment target.

2. The system of claim 1, wherein the user data is received from multiple unstructured data sources.

3. The system of claim 1, wherein the user data is patient data.

4. The system of claim 1, wherein the at least one AI model includes at least one of a machine learning model and a natural language processing model.

5. The system of claim 1, wherein the at least one sentiment target is at least one of:
a pharmaceutical, a medical therapy, educational materials, an AI assistant, policies and regulations, an insurance claim, a healthcare platform, mental health, and a health program campaign.

6. The system of claim 1, wherein the at least one output includes a sentiment analysis index score for at least one sentiment target.

7. The system of claim 6, wherein multiple sentiment analysis index scores are provided for each sentiment target.

8. The system of claim 7, wherein each sentiment analysis score represents a specific demographic subset of the user data.

9. The system of claim 1, wherein the at least one output includes predictive analytics for an outcome associated with the sentiment target.

10. The system of claim 9, wherein the predictive analytics provide predictions for at least one of: patient adherence, drug efficacy, adverse drug reactions, compliance, and fraud detection.

11. A method of analyzing a sentiment of user data by a sentiment analysis engine comprising at least one processor and at least one non-transitory memory storing processor-executable instructions, the method comprising:
receiving, at the sentiment analysis engine, user data from at least one source and at least user;
analyzing, by at least on AI model, the user data to identify a sentiment of the user data for at least one sentiment target, based on keywords and language found within the user data; and
generating at least one output indicating the sentiment of the user data for the at least one sentiment target.

12. The method of claim 11, wherein the user data is received from multiple unstructured data sources.

13. The method of claim 11, wherein the user data is patient data.

14. The method of claim 11, wherein the at least one AI model includes at least one of a machine learning model and a natural language processing model.

15. The method of claim 11, wherein the at least one sentiment target is at least one of: a pharmaceutical, a medical therapy, educational materials, an AI assistant, policies and regulations, an insurance claim, a healthcare platform, mental health, and a health program campaign.

16. The method of claim 11, wherein generating the at least one output includes generating a sentiment analysis index score for at least one sentiment target.

17. The method of claim 16, wherein multiple sentiment analysis index scores are provided for each sentiment target.

18. The method of claim 17, wherein each sentiment analysis score represents a specific demographic subset of the user data.

19. The method of claim 11, wherein generating the at least one output includes generating predictive analytics for an outcome associated with the sentiment target.

20. The method of claim 19, wherein the predictive analytics provide predictions for at least one of: patient adherence, drug efficacy, adverse drug reactions, compliance, and fraud detection.
